(19) United States
(12) Patent Application Publication
(10) Pub. No.: US 2006/0019882 A1
(43) Pub. Date: Jan. 26, 2006

(54) Use of Yops as caspase inhibitor

(75) Inventors: Rudi Beyaert, Zingem (BE); Peter Schotte, Ettelgem (BE)

Correspondence Address: TRASK BRITT, P.O. BOX 2550, SALT LAKE CITY, UT, 84110, US

(73) Assignees: Vlaams Interuniversitair Instituut Voor Biotechnologie VZW, Zwijnaarde (BE); Universiteit Gent, Gent (BE)

(21) Appl. No.: 11/184,769

(22) Filed: Jul. 19, 2005

Related U.S. Application Data
(63) Continuation of application No. PCT/EP04/50026, filed on Jan. 20, 2004.

(30) Foreign Application Priority Data
Jan. 20, 2003 (EP) 03100106.8

(51) Int. Cl. A61K 38/16

(52) U.S. Cl. 514/8

(57) ABSTRACT

The present invention relates to the use of Yops as caspase inhibitors. More specifically, it relates to the use of YopE and YopT as inhibitors of caspase-1 activity. The inhibitor can be used to treat caspase-1-related pathologies, such as inflammatory diseases and to inhibit caspase-1-related and/or -mediated cell death.

### CROSS-REFERENCE TO RELATED APPLICATIONS

This application is a continuation of PCT International Patent Application No. PCT/EP2004/050026, filed on Jan. 20, 2004, designating the United States of America, and published, in English, as PCT International Publication No. WO 2004/064713 A2 on Aug. 5, 2004, which application claims priority to European Patent Application Serial No. 03100106.8 filed on Jan. 20, 2003, the contents of the entirety of each of which are incorporated by this reference.

### TECHNICAL FIELD

The present invention relates to the use of Yops (Yersinia outer membrane proteins) as caspase inhibitor. More specifically, it relates to the use of YopE and YopT as inhibitor of the caspase-1 activity. The inhibitor can be used to treat caspase-1-related pathologies such as inflammatory diseases, or to inhibit caspase-1-related cell death.

### BACKGROUND

A number of Gram-negative pathogens subvert the innate immune system of their host by a virulence mechanism called type III secretion system (TTSS). In the archetypal Yersinia—Y. pestis, agent of bubonic plague, Y pseudotuberculosis and Y. enterocolitica—the TTSS is encoded on a 70-kb virulence plasmid (Cornelis et al., 1998). By this mechanism, Yersinia bacteria adhering at the surface of eukaryotic cells inject proteins—called Yops—across cellular membranes into the cytosol of these cells. These Yops are powerful “effectors” that take control of the host cells by hijacking the intracellular machinery (Cornelis, 2002). YopE, YopT, YopO and YopH cooperatively lead to the destruction of the actin cytoskeleton and by doing so prevent phagocytosis. Besides its anti-phagocytic role, YopH also prevents the release of the macrophage chemoattractant MCP-1 by blocking the phosphatidylinositol-3 kinase pathway (Sauvonnet et al., 2002). YopP has been shown to induce the rapid generation of pro-apoptotic tBid (Denecker et al., 2001). In addition, YopP binds to and prevents the activation of members of the MAP kinase kinase family and of IκB kinase β (Orth et al., 1999). In this way YopP efficiently shuts down NF-κB-dependent signaling pathways, preventing survival signaling and the production of pro-inflammatory cytokines such as TNF and IL-8. IL-1β is a pleiotropic cytokine that is involved in the regulation of both the innate and acquired immune response (Fitzgerald and O'Neill, 2000). IL-1β expression in macrophages is inducible in a NF-κB-dependent way (Goto et al., 1999), and it is synthesized as inactive precursor whose maturation is controlled by the cysteine protease caspase-1 (Howard et al., 1991). The latter is present in the cytosol as a 45-kDa precursor, which undergoes a series of processing events eventually leading to the formation of a (p20/p10)₂ heterotetramer (Wilson et al., 1994). Secretion of IL-1β does not occur through the classical endoplasmic reticulum-Golgi network, and evidence has been presented that caspase-1 may be a component of the secretory apparatus localized on the external cell surface membranes (Kuida et al., 2000; Li et al., 1995; Singer et al., 1995; MacKenzie et al., 2001).

### SUMMARY OF THE INVENTION

Surprisingly, we found that Yops can act as inhibitors of caspase. More specifically, YopE and YopT can act as an inhibitor of caspase-1. Even more surprisingly, we were able to demonstrate that YopE and YopT execute their inhibitory function on caspase-1 by their effect on Rho GTPase, and that Rho GTPase plays a role in the regulation of caspase-1 activity. In an embodiment, Rho GTPase activates caspase-1 by involvement of LIMK-1. In an alternate embodiment, Rho GTPase is Rac1.

A first embodiment of the invention is the use of a Yop (Yersinia outer membrane protein) effector protein as caspase inhibitor. In an embodiment, inhibition is prevention of the caspase oligomerization. In an alternate embodiment, caspase oligomerization is Asc-induced caspase oligomerization. In various embodiments, the Yop effector protein is YopE and/or YopT, and the caspase is caspase-1.

Another embodiment of the invention is the use of a Yop effector protein to treat caspase-related diseases. In an embodiment, the Yop effector protein is YopE and/or YopT, and the caspase is caspase-1. Caspase-related diseases and especially caspase-1-related diseases are, as a non-limiting example, inflammatory bowel disease, Crohn's disease, rheumatoid arthritis, chronic pancreatitis, multiple sclerosis, Alzheimer disease, Huntington's disease and metastatic melanoma. Indeed, for all these diseases, it is known that expression of caspase-1, or the level of mature interleukin-1 as a result of the processing by caspase-1, plays an essential role.

Still another embodiment of the invention is the use of a Yop effector protein to inhibit caspase-1-mediated cell death. In an embodiment, the Yop effector protein is YopE and/or YopT.

Still another embodiment of the invention is the use of a Yop effector protein to inhibit Interleukin-1β and/or interleukin-18 maturation. In an embodiment, the Yop effector protein is YopE and/or YopT. Indeed, it is known that the maturation of those molecules is caspase-1 mediated, and therefore, inhibition of caspase-1 results in inhibition of the maturation of interleukins.

Still another embodiment of the invention is the use of a Yop effector protein to inhibit Interleukin-1β and/or interleukin-18 release from cells. In an embodiment, the Yop effector protein is YopE and/or YopT.

Another embodiment of the invention is the use of a Rho GTPase to modulate caspase-1 activity. In an embodiment, modulation is a modulation of the oligomerization of caspase-1. In an embodiment, modulation is a modulation of the Asc-induced caspase-1 oligomerization. Rho GTPases are known to the person, skilled in the art, and have been described, amongst others, by Etienne-Manville and Hall, Nature, 420, 629-635, 2002. In an embodiment, Rho GTPase is RhoA, Rac1 or Cdc42.

Still another embodiment of the invention is the use of a Rho GTPase inhibitor to inhibit caspase-1 activation and/or activity. Indeed, as in this invention it was demonstrated for the first time that Rho GTPase plays an essential role in caspase-1 activation, it is evident that Rho GTPase inhibitors can be used to block caspase-1 activation.

As a consequence, another embodiment of the invention is the use of a Rho GTPase inhibitor to treat caspase-1-related diseases.

Another embodiment of the invention is the use of a Rho GTPase inhibitor to inhibit caspase-1-mediated cell death.

Still another embodiment of the invention is the use of a Rho GTPase inhibitor to inhibit interleukin-1β and/or interleukin-18 maturation.

Still another embodiment of the invention is the use of a Rho GTPase inhibitor to inhibit interleukin-1β and/or interleukin-18 release from cells.

Rho GTPase inhibitors are known to the person skilled in the art, and include, but are not limited to geranylgeranyl protein transferase inhibitors and farnesyl protein transferase inhibitors.

Another embodiment of the invention is the use of LIMK-1 to modulate caspase-1 activity. In an embodiment, modulation is a modulation of the oligomerization of caspase-1.

Still another embodiment of the invention is the use of a LIMK-1 inhibitor to inhibit caspase-1 activation and/or activity. In an embodiment, inhibition of the activation is an inhibition of the oligomerization of caspase 1. In an alternate embodiment, inhibition is an inhibition of the Asc-induced oligomerization of caspase-1.

As a consequence, another embodiment of the invention is the use of a LIMK-1 inhibitor to treat caspase-1-related diseases.

Another embodiment of the invention is the use of a LIMK-1 inhibitor to inhibit caspase-1-mediated cell death.

Still another embodiment of the invention is the use of a LIMK-1 inhibitor to inhibit interleukin-1β and/or interleukin-18 maturation.

Still another embodiment of the invention is the use of a LIMK-1 inhibitor to inhibit interleukin-1β and/or interleukin-18 release from cells.

The study of host-pathogen interactions revealed eukaryotic cell processes not understood before. In the present invention, we have demonstrated a new role for the Yersinia effector proteins YopE and YopT in down-regulating the inflammatory response, and we have highlighted a previously unknown function of Rho GTPases in the activation of caspase-1 and the release of IL-1β. As for its anti-phagocytic defense, Yersinia seems to inhibit the production of pro-inflammatory cytokines by a complex interplay between several Yop effectors that act at multiple levels. Intriguingly, modulation of caspase-1-mediated inflammation might also occur during infection with several other pathogens such as Pseudomonas spp., Clostridium spp., Salmonella spp., Bacillus spp. and Staphylococcus spp., which encode proteins that are also known to target specific Rho GTPases. Our findings may therefore give new insights in drug design for treating infectious diseases.

### BRIEF DESCRIPTION OF THE FIGURES

FIG. 1. YopE inhibits the release of IL-1β in Y. enterocolitica-infected macrophages. Panel A: Mf4/4 macrophages were infected with wild-type (WT) or YopP-deficient (YopP) derivatives of Y. enterocolitica E40. Non-infected (NI) Mf4/4 cells were used as a control. Two hours after the beginning of infection, gentamicin (50 μg/ml) was added to kill extracellular bacteria. Four hours later, cell supernatants were collected and cytosolic cell lysates were prepared. IL-1β or IL-6 release in the supernatant was analyzed by ELISA specific for IL-1β (Quantikine, R&D Systems) or IL-6 (Biotrak, Pharmacia Biotech). Panel A, inset: Cytosolic proteins were subjected to SDS-PAGE and transferred to Hybond nitrocellulose membranes (Pharmacia Biotech). The blot was probed with polyclonal antibodies against IL-1β. Panel B: Mf4/4 cells were infected with WT, a virulence plasmid-negative strain (PYV) or Yersinia enterocolitica strains deficient for one or multiple Yop effector proteins, as indicated, and analyzed for IL-1β release as described in Panel A. Panel C: Mf4/4 cells were infected with YopPE⁻, or YopPE⁻ strains that were complemented with wild-type (YopEWT) or mutant YopE (YopE_{M}) as indicated, and analyzed for IL-1β release as described in Panel A. The lower part of Panel C shows the intracellular expression levels of proIL-1β as revealed by Western blot analysis of the corresponding cell lysates, and the relative LDH activity released into the medium of infected cells compared to non-infected cells as revealed by the Cytotox-one homogeneous membrane integrity assay (Promega). Panel D: Mf4/4 cells were infected as described under Panel C and TCA-precipitated proteins in the medium were analyzed by Western blot analysis for the presence of proIL-1β and mature IL-1β (* indicates a non-specific band).

FIG. 2. Specific Yop effector proteins and Rho GTPases regulate procaspase-1 activation. Panel A: HEK293T cells were transiently transfected with expression plasmids encoding procaspase-1 (100 ng), proIL-1β (200 ng), β-galactosidase and 4 ng of either empty vector (EV) or an expression vector encoding wild-type (WT) YopE, YopT or YopH or the catalytically inactive mutants (M) YopE_{R144A} or YopT_{C139S}, all fused N-terminally with an E-tag. Cell lysates were subjected to SDS-PAGE and transferred to Hybond nitrocellulose membranes. The blot was probed with polyclonal antibodies against caspase-1 and reprobed with anti-IL-1β. p20 and p22 represent specific processing products of caspase-1. The expression of YopE, -T and -H was confirmed using the monoclonal anti-E-tag-HRP antibody. Panels B-E: HEK293T cells were transiently transfected with the indicated amounts of expression vectors for the constitutive-active (CA) mutants RhoA_{Q63L} (RhoA_{CA}), Rac1_{G12V} (Rac1_{CA}) or Cdc42_{Q61L} (Cdc42_{CA}), or an expression vector for the dominant-negative (DN) mutant Rac1_{T17N} (Rac1_{DN}), together with expression plasmids encoding procaspase-1 (100 ng), proIL-1β (200 ng), and β-galactosidase. Cell death was measured 24 hours after transfection by the release of β-galactosidase into the medium using the Galactostar reporter gene assay system (Tropix, Applera Belgium N.V.) and values are expressed as % of the amount that was released into the medium of cells that were only transfected with procaspase-1 and proIL-1β. Secretion of mature bio-active IL-1β (Panels B-D) was assayed 24 hours after transfection in a CTLL cell proliferation assay (Vandenabeele et al., 1990). Values are expressed in pg/ml and corrected for transfection efficiency as reflected by β-galactosidase activity measured in the corresponding cell lysates. Standard deviation (n=3) was <10%. Cell extracts were subjected to SDS-PAGE and transferred to Hybond nitrocellulose membranes. Expression of transfected Rho GTPases was confirmed by Western blotting using the monoclonal anti-E-tag-HRP antibody. To detect caspase-1 processing (Panels C and E), the blot was probed with polyclonal antibodies against caspase-1.

FIG. 3. Rho GTPase inhibitors prevent caspase-1 auto-activation and IL-1β maturation. HEK293T cells were transfected with expression plasmids for procaspase-1 (100 ng) and proIL-1β (200 ng). Medium was replaced four hours after transfection with medium containing GGTI-2147 (10 μM, Calbiochem) or Toxin B (10 pM, Calbiochem). Cells were lysed 24 hours after transfection and proteins were subjected to SDS-PAGE and transferred to Hybond nitrocellulose membranes. The blot was probed with polyclonal antibodies against caspase-1 (upper panel) and reprobed with anti-IL-1β (lower panel).

FIG. 4. Rac1 signaling towards LIMK-1 but not JNK is required for Rac1-mediated activation of caspase-1. Panel A: HEK293T cells were transiently transfected with empty vector (EV) or an expression vector (50 ng) encoding the constitutive-active (CA) mutants Rac1^{CA}, Rac1_{CA-F37L} or Rac1_{CA-Y40H}, together with expression plasmids encoding procaspase-1 (100 ng), proIL-1β (200 ng) and a β-galactosidase reporter plasmid. Panel B: HEK293T cells were transiently transfected with an expression vector (50 ng) encoding procaspase-1 together with empty vector (EV) or the indicated amounts of expression plasmids encoding the constitutive-active or dominant negative mutants of LIMK-1 and constitutive-active (50 ng) Rac1_{CA}. Cell extracts were subjected to SDS-PAGE and transferred to Hybond nitrocellulose membranes. The blot was probed with polyclonal antibodies against caspase-1. Expression of Rac1 and LIMK-1 was confirmed using the monoclonal anti-E-tag-HRP or an anti-myc-tag antibody, respectively. Supernatant was harvested 24 hours after transfection of HEK293T cells, and secretion of mature bio-active IL-1β was assayed in a CTLL cell proliferation assay. Values are expressed in pg/ml and corrected for transfection efficiency as reflected by β-galactosidase activity measured in the corresponding cell lysates. Standard deviation (n=3) was <10%.

FIG. 5. Asc-mediated activation of caspase-1 is modulated by Rac1. Panel A: HEK293T cells were transiently transfected with an expression vector (50 ng) encoding procaspase-1 and Asc (50 ng) together with empty vector (EV) or with expression plasmids encoding YopT, YopE or the dominant-negative mutants of LIMK-1 or Rac1_{DN}. Panel B: HEK293T cells were transiently transfected with an expression vector (50 ng) encoding procaspase-1 and Asc (20 ng) as indicated together with empty vector (EV) or with an expression plasmid encoding Rac1_{CA} (20 ng). Cell extracts were subjected to SDS-PAGE and transferred to Hybond nitrocellulose membranes. The blot was probed with polyclonal antibodies against caspase-1. Expressions of Rac1, YopE and YopT were confirmed using the monoclonal anti-E-tag-HRP antibody. The expression of LIMK-1 and Asc were confirmed using a monoclonal anti-myc or an anti-Flag-HRP antibody, respectively. Supernatant was harvested 24 hours after transfection and secretion of mature bio-active IL-1β was assayed in a CTLL cell proliferation assay. Values are expressed in pg/ml and corrected for transfection efficiency as reflected by β-galactosidase activity measured in cell lysates. Standard deviation (n=3) was <10%.

FIG. 6. Rho GTPases regulate the dimerization of procaspase-1. Co-immunoprecipitation assays were performed using lysates from HEK293T cells that were transiently transfected with plasmids (100 ng) encoding enzymatically inactive E-tagged procaspase-1 (C284A) and Flag-tagged procaspase-1 (C284A) as indicated with E or F respectively, and 20 ng of either empty vector (EV) or an expression vector encoding YopE, YopT, Rac1_{DN} or Rac1_{CA}. Immunoprecipitates were prepared using anti-Flag antibody adsorbed to protein G-sepharose and analyzed by SDS-PAGE/immunoblotting using anti-E-tag-HRP antibody. Expressions of Rac1, YopE and YopT were confirmed using the monoclonal anti-E-tag-HRP antibody.

### DETAILED DESCRIPTION OF THE INVENTION

### Examples

Materials and Methods to the Examples

Plasmids and antibodies. Wild-type (WT) YopE, YopT and YopH were amplified by polymerase chain reaction from the E40(pYV40) plasmid (Sory et al., 1995) and cloned in frame with an N-terminal E-tag into pCAGGS-Etag (Heyninck et al., 1999) cut with NotI and XmaI restriction enzymes. The inactive mutants (M) YopE_{R144A} and YopT_{C139S} were generated by overlapping polymerase chain reaction using mutated primers. pYV40 plasmids for specific Yop effector knockouts have been described previously (Iriarte and Cornelis, 1998; Mills et al., 1997). Expression plasmids for YopE_{WT} and YopE_{M}, which were used for complementation of YopE knockout strains, were a gift from Dr. L. J. Mota. The cDNA of human RhoA, Rac1, Cdc42 and their corresponding dominant-negative (T¹⁷→N¹⁷ in Rac1 and Cdc42, T¹⁹→N¹⁹ in RhoA) and constitutive-active (Q⁶¹→L⁶¹ in Cdc42, Q⁶³→L⁶³ in RhoA, G¹²→V¹² in Rac1) mutants have been described previously (Sander et al., 1999), and were a kind gift of Dr. J. Piette. The cDNA of Rho family members and caspase-1 were amplified by polymerase chain reaction and cloned in frame with an N-terminal E-tag or Flag-tag into pCAGGS (Niwa et al., 1991) cut with NotI and XmaI restriction enzymes. Overlapping polymerase chain reaction using constitutive-active Rac1 as a template and mutated primers generated the constitutive-active mutants Rac1_{CA-F37L} and Rac1_{CA-Y40H}, respectively. The mouse proIL-1β cDNA was cloned into pCAGGS-Etag vector with an additional HA-tag at the 3′-end of the proIL-1β cDNA. All constructs were confirmed by DNA sequence analysis. The expression plasmid for N-terminal Flag-tagged human Asc (pCR3.V66-Met-Flag-Asc) and myc-tagged LIMK-1 constructs were kind gifts of Dr. Jurg Tschopp and Dr. Pico Caroni (Basel, Switzerland), respectively. The β-galactosidase-encoding plasmid pUT651 was purchased from Cayla (Toulouse, France). A rabbit polyclonal antibody against recombinant murine caspase-1 was prepared by the Centre d'Economie Rurale (Laboratoire d'Hormonologie Animale, Marloie, Belgium).

Bacterial strains and growth conditions. Escherichia coli Top10 or MC1061 were used for standard manipulations; E. coli SM10 lambda pir₊ was used to deliver mobile plasmids into Y. enterocolitica (Cornelis et al., 1998). E. coli strains were routinely grown at 37° C. in tryptic soy broth or on tryptic soy agar plates containing the appropriate antibiotics. Y enterocolitica bacteria were grown at 25° C. in brain-heart infusion (BHI; Difco) or on tryptic soy agar plates containing the appropriate antibiotics. Y. enterocolitica E40 strains and derivatives have been described before (Iriarte and Cornelis, 1998; Mills et al., 1997). For infections, bacteria were diluted to an OD 0.1 in fresh BHI medium and incubated at 25° C. for 120 minutes. Subsequently, Yop secretion was induced by incubation for 30 minutes in a shaking water bath (110 rpm) at 37° C. Prior to infection bacteria were washed with RPMI1640.

Culture, infection and transfection of cells. The murine macrophage cell line Mf4.4 (Desmedt et al., 1998), and the human embryonic kidney cell line HEK293T were cultured at 37° C. in RPMI1640 or DMEM, respectively, supplemented with 10% FBS, 2 mM L-glutamine, penicillin (100 U/ml), streptomycin sulphate (100 μg/ml), sodiumpyruvate (1 mM) and β-mercaptoethanol (2×10⁻⁵ M). Prior to infection, Mf4/4 cells were seeded in medium without antibiotics. After 15 hours, cells were infected at a multiplicity of infection (m.o.i.) of 50 with the relevant Y. enterocolitica strains that were grown at 37° C. under conditions for moderate Yop induction (see above). Extracellular bacteria were killed two hours after infection by adding gentamicin (50 μg/ml). HEK293T cells were plated in 6-well plates at 2×10⁵ cells per well and transiently transfected by calcium phosphate-DNA coprecipitation. Twenty-four hours after transfection, medium was removed and cells were lysed in 300 μl lysis buffer (50 mM Hepes, pH 7.6, 200 mM NaCl, 0.1% NP40, 5 mM EDTA). Proteins were separated by SDS-PAGE and analyzed by Western blotting with rabbit polyclonal anti-caspase-1 and anti-IL-1 β antibodies (R&D systems), respectively, with mouse monoclonal anti-FlagHRP (Sigma-Aldrich) or anti-E-tagHRP antibodies (Pharmacia Biotech). Immunoreactivity was revealed with the enhanced chemiluminescence method (NEN™ Renaissance, NEN Life Sciences Products). LDH release was assayed using Cytotox-one homogeneous membrane integrity assay as described by the manufacturers protocol (Promega). β-Galactosidase release was assayed using the Galactostar reporter gene assay system (Tropix, Applera Belgium N.V.)

### Example 1

### YopE Inhibits the Release of IL-1β in Y. enterocolitica-Infected Macrophages

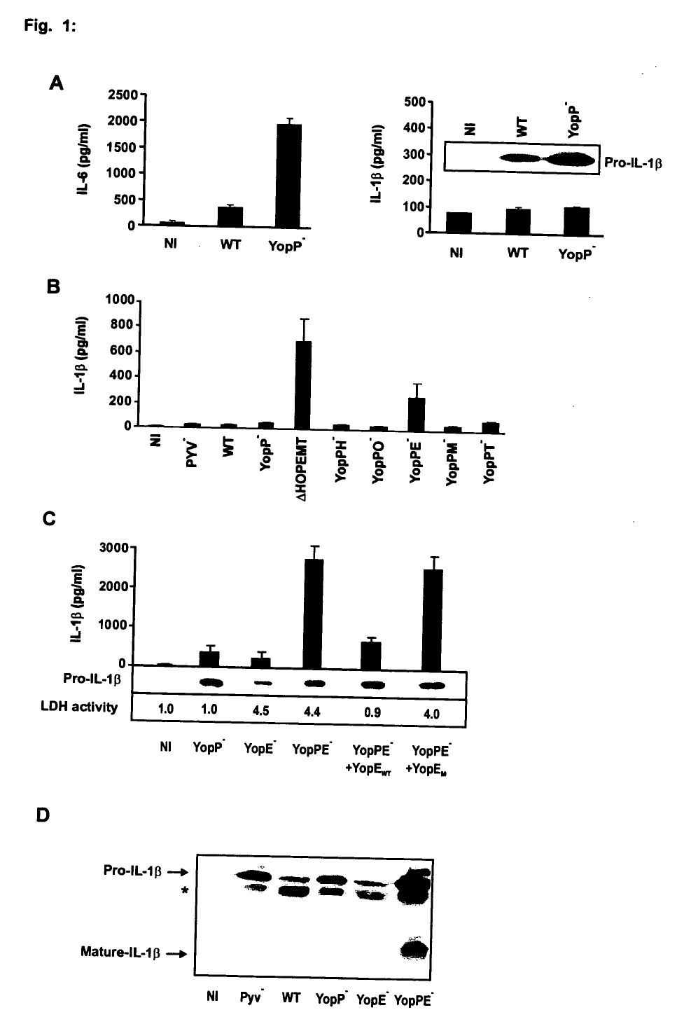

Yersinia has previously been shown to prevent NF-κB activation in infected cells in a YopP-dependent manner (Ruckdeschel et al., 2001). Therefore, it could be expected that the expression of NF-κB-dependent genes would be strongly increased in cells infected with a YopP-deficient strain (YopP⁻) compared to cells infected with wild-type (WT) Yersinia. To verify this, we compared the amount of IL-1β and IL-6 in the supernatant of Mf4/4 macrophages that were infected with either YopP⁻ or WT Yersinia enterocolitica. To our surprise, only the levels of IL-6 but not those of IL-1β were increased in the supernatant of YopP⁻-infected cells (FIG. 1, Panel A). Nevertheless, intracellular IL-1β levels in the corresponding cell lysates were considerably higher in cells infected with the YopP⁻ strain (FIG. 1, Panel A, inset). It should be mentioned that only the precursor form of IL-1β could be detected in these cell extracts. The above observations indicated a role for another Yop effector protein in the regulation of IL-1β maturation and its release in the cell supernatant. Therefore, we infected macrophages with a Y. enterocolitica strain that is deficient for all six Yop effectors (ΔHOPEMT, FIG. 1, Panel B), which indeed resulted in a large increase of IL-1β levels in the supernatant (FIG. 1, Panel B). All together, the above results demonstrate that, besides the YopP-mediated down-regulation of IL-1β production at the transcriptional level, other Yop effectors could specifically inhibit the maturation and release of IL-1β. To analyze which Yop effector controls the release of IL-1β, we constructed five double knockout Y. enterocolitica strains, which are negative for YopP and for any one of the five other Yops: YopPE⁻, YopPT⁻, YopPO⁻, YopPH⁻ and YopPM⁻ strains. After infection of macrophages with each of these double-deficient bacteria, we analyzed again the secretion of IL-1β in the supernatant. IL-1β release was still strongly inhibited in macrophages infected with the double mutant bacteria YopPT⁻, YopPO⁻, YopPH⁻ and YopPM⁻, although a small but reproducible increase in IL-1β could be detected in YopPT⁻-infected cells (FIG. 1, Panel B). However, cells infected with the YopPE⁻ train released significantly higher levels of IL-1β, suggesting that YopE is at least partially responsible for the inhibition of IL-1β release as seen upon infection with WT or YopP-deficient Yersinia. Since IL-1 levels released by YopPE⁻ and YopPT⁻-infected cells were still lower than those released from cells infected with the ΔHOPEMT mutant, one could expect that cells infected with a triple YopPET mutant would release comparable amounts of IL-1β as those infected with the ΔHOPEMT mutant. However, we were unable to see a reproducible difference between cells infected with YopPET⁻ or YopPE⁻ mutants. As shown in FIG. 1, Panel B, infection of macrophages with a Yersinia strain (PYV⁻), which forms no functional secretion apparatus, does not induce caspase-1 activation. This suggests that the initial signal for caspase-1 activation may be initiated from one or more components of the secretion apparatus. A possible hypothesis could be that the secretion apparatus activates Rho GTPases which may result in cytoskeletal rearangements leading to intracellular K⁺ loss and activation of the inflammasome.

YopE is a GAP for Rho GTPases, in particular Rac1 (Andor et al., 2001), switching them off by accelerating GTP hydrolysis (Von Pawel-Rammingen et al., 2000). To analyze if the GAP activity of YopE is required for inhibition of IL-1β release, we complemented the YopPE⁻ strain with wild-type YopE (YopE_{WT}) or with a mutant of YopE (YopE_{M}) that lacks the GAP activity (FIG. 1, Panel C). Complementation of the YopPE⁻ strain with YopE_{WT}, but not with YopE_{M}, restored the potential of Yersinia to prevent the release of IL-1β in the medium of infected macrophages. In contrast, intracellular expression levels of proIL-1β were independent of YopE (FIG. 1, Panel C). A previous report showed that installation of the Yersinia secretion apparatus into the cell membrane of the macrophage results in pore formation and the release of cytosolic proteins such as lactate dehydrogenase (LDH), which could be prevented by YopE. Moreover, a role for Rho GTPases and rearrangements of the cytoskeleton in the regulation of this pore formation was illustrated (Viboud and Bliska, 2001). In agreement with the latter observation, YopE deficiency also resulted in a significant increase of LDH and proIL-1β release in our experiments (FIG. 1, Panels C and D, respectively), which coincided with an apoptotic morphology of the cells. However, also a clear band corresponding to mature active IL-1β could be seen in the supernatant of YopPE-infected cells, implicating that the increased IL-1β levels measured in the ELISA experiments are not just due to an increase in proIL-1β release. The latter was further confirmed by the fact that identical results could be obtained when the IL-1 levels in the supernatant were measured with a CTLL cell proliferation assay that detects specifically mature bio-active IL-1β (Vandenabeele et al., 1990). These results indicate that the GAP activity of YopE is responsible for the inhibition of the maturation and secretion of IL-1β in Yersinia-infected macrophages. Moreover, this also implies a role for Rho GTPases in the process of IL-1β maturation and IL-1β release.

### Example 2

### Specific Yop Effector Proteins and Rho GTPases Regulate Procaspase-1 Activation

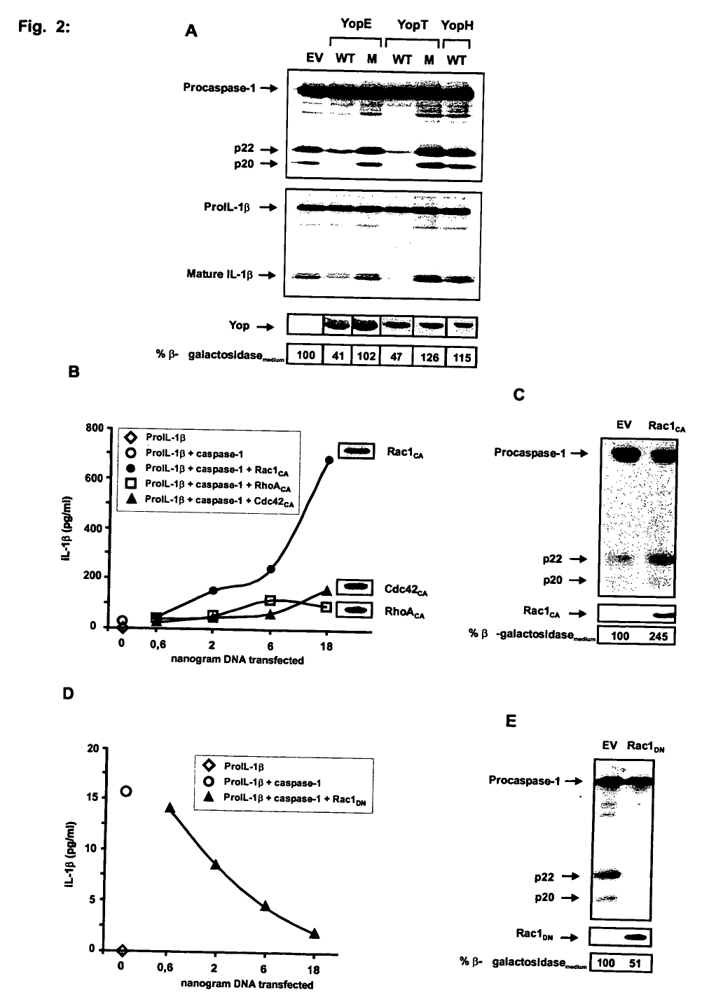

Because IL-1β maturation is mediated by caspase-1, we next analyzed the effect of YopE on caspase-1-mediated IL-1β maturation in HEK293T cells that were transiently transfected with procaspase-1 and proIL-1β. In these conditions, overexpression of procaspase-1 induces its autocatalytic processing to an active p20/p10 form, resulting in the maturation of the 33 kDa proIL-1β to the bio-active 17 kDa form (FIG. 2, Panel A). Moreover, caspase-1 auto-activation also results in the induction of cell death, which can be assayed by the release of cotransfected β-galactosidase into the medium (FIG. 2, Panels A, C and E). Additional co-expression of YopE_{WT}, but not of the catalytically inactive mutant YopE_{M}, inhibited the autocatalytic processing of procaspase-1 as well as the proteolytic maturation of proIL-1β. Furthermore, also β-galactosidase release into the medium was partially inhibited. This demonstrates that YopE can interfere with the autocatalytic processing of caspase-1 through its GAP activity, leading to a decrease in proIL-1β maturation and caspase-1-mediated cell death. Interestingly, co-expression of YopT had a similar inhibitory effect on the proteolytic activation of procaspase-1 and proIL-1β (FIG. 2, Panel A). YopT functions as a cysteine protease that cleaves off the prenylated C-terminus of Rho, Rac and Cdc42, leading to their release from the plasma membrane and their irreversible inactivation (Shao et al., 2002). A mutant of YopT (YopT_{M}) that has lost its proteolytic activity on Rho GTPases, was no longer able to prevent the processing of procaspase-1 and proIL-1β. In contrast to YopE and YopT, co-expression of the effector protein YopH, which is a tyrosine phosphatase that has been shown to dephosphorylate proteins from focal adhesions and other signaling complexes (Black and Bliska, 1997; Black et al., 2000; Persson et al., 1997), had no effect on caspase-1 activity. Although YopH can lead to a rearrangement of the actin cytoskeleton (Grosdent et al., 2002), an effect on Rho GTPases has not been reported. These experiments show that YopE and YopT can interfere with the autocatalytic processing and the activation of caspase-1 by interfering with the function of Rho GTPases. The inhibitory effect of YopT expression on IL-1β maturation is somewhat contradictory to the lack of a significant increase in IL-1β release in cells infected with a YopPT⁻ double knockout as described in our previous experiments (FIG. 1, Panel B). Most likely, this can be explained by a difference in the Yop concentration in cells upon overexpression or infection, respectively. In this context, it should be mentioned that whereas YopE and YopT inactivate RhoA, Rac 1, and Cdc42 in vitro (Andor et al., 2001; Shao et al., 2002), when they are injected into cells by Yersinia, they are remarkably specific, inactivating selectively Rac1 and RhoA, respectively (Aepfelbacher et al., 2003; Andor et al., 2001). These results suggest a major role for Rac1 in caspase-1 activation. In order to confirm the role of Rho GTPases, and in particular Rac1, in the proteolytic activation of caspase-1, HEK293T cells were cotransfected with procaspase-1, proIL-1β, β-galactosidase, and constitutive-active (CA) mutants of RhoA, Rac1 and Cdc42. We hypothesized from our previous experiments that the constitutive-active Rho GTPases should promote the autocatalytic processing of procaspase-1 and the corresponding maturation and secretion of IL-1β. Indeed, IL-1β levels were significantly increased in the supernatant of cells overexpressing RhoA_{CA}, Cdc42_{CA} or Rac1_{CA}. Titration of the transfected plasmid DNA concentration clearly showed that Rac1_{CA} is much more efficient then RhoA or Cdc42 (FIG. 2, Panel B), which is in agreement with the more pronounced release of IL-1β in YopPE⁻ versus YopPT⁻-infected cells (FIG. 1, Panel B) and the described preferential effect of YopE on Rac1 (Andor et al., 2001). As expected, Rac1_{CA} also enhanced the autoproteolytic activation of procaspase-1 and the release of β-galactosidase (FIG. 2, Panel C), whereas RhoA_{CA} and Cdc42_{CA} did not. Reversely, transfection of a dominant-negative mutant of Rac1 (Rac1_{DN}) resulted in a decrease of caspase-1 auto-activation and, consequently, in a decrease of IL-1β and β-galactosidase release (FIG. 2, Panels D and E). It should be mentioned that the ectopic expression levels of Rac1 that are needed to affect caspase-1 activation are extremely low, being under the detection limit in the case of Rac1_{DN} (FIG. 2, Panel E). All together, the above observations imply an important role for Rho GTPases, especially Rac1, in the regulation of caspase-1 activity.

### Example 3

### Pharmaceutical Modulation of Rho GTPase Activity Affects Caspase-1 Activation and IL-1β Maturation

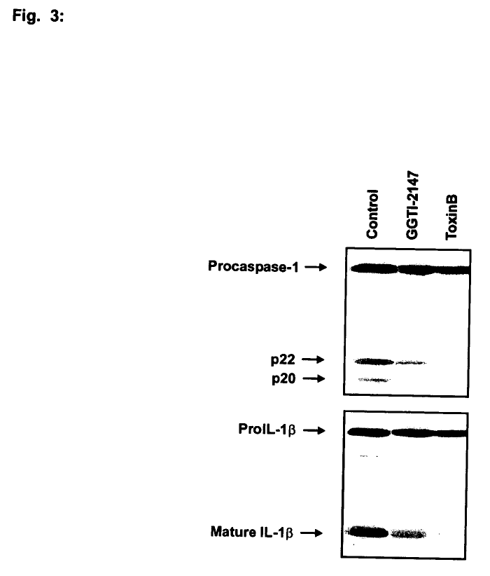

To further confirm the role of Rho GTPases in caspase-1 activation and IL-1β production, we analyzed the effect of Clostridium difficile Toxin B. The latter is a glucosyltransferase that covalently links a glucose moiety on a critical threonine residue of Rho, Rac and Cdc42 (Prepens et al., 1996; Wilkins and Lyerly, 1996), thus impairing the docking of the GTPases on their effectors. Similarly, we also tested the effect of the geranylgeranyl transferase inhibitor GGTI-2147, which prevents the prenylation and membrane localization of Rho GTPases (Vasudevan et al., 1999). Western blot analysis revealed that treatment of procaspase-1 and proIL-1β-expressing HEK293T cells with Toxin B or GGTI-2147 significantly prevents the proteolytic auto-activation of caspase-1 and maturation of proIL-1β (FIG. 3). These results further demonstrate that Rho GTPases play an important role in the regulation of caspase-1 activation, and the corresponding caspase-1-mediated maturation and release of IL-1 β.

### Example 4

### The Effect of Rac1 on Caspase-1 Activation is Independent of its Effect on the JNK Pathway, but is Mediated by LIM Kinase-1

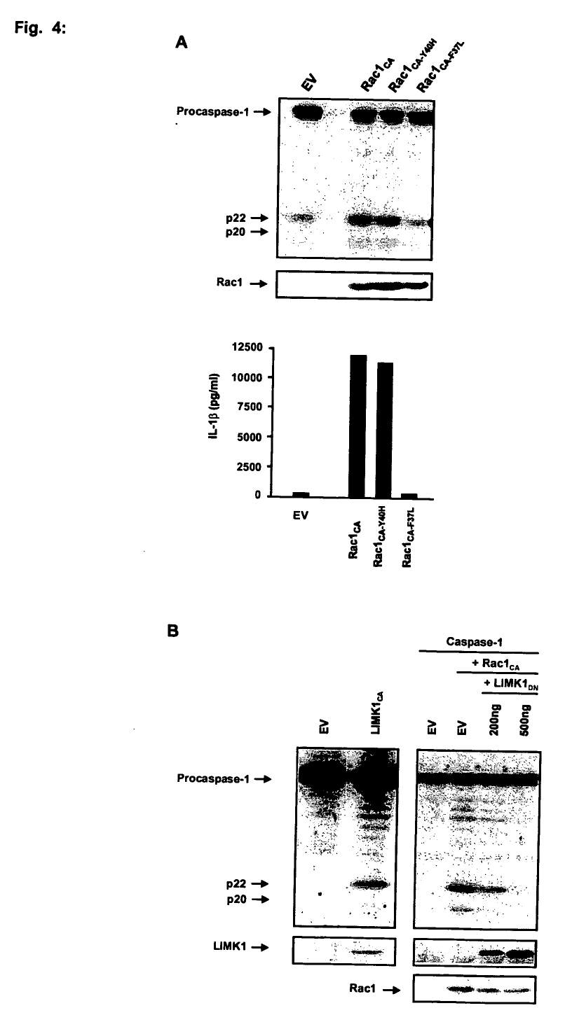

The functions of Rho GTPases, first assigned to the regulation of the organization of the actin cytoskeleton, have been extended to many other cellular processes, including activation of the c-Jun N-terminal kinase (JNK) (Bishop and Hall, 2000). Moreover, Rac-induced cytoskeleton reorganization and JNK activation are the result of independent Rac-induced signaling pathways (Lamarche et al., 1996; Westwick et al., 1997). To dissect which signaling pathway is important in the Rac1-induced-activation of caspase-1, we used specific point mutants of Rac1_{CA} that are defective in either JNK activation (Rac1_{CA-Y40H}) or actin reorganization (Rac1_{CA-F37L}) (Lamarche et al., 1996; Westwick et al., 1997). Transfection of cells with Rac1_{CA} or Rac1_{CA-Y40H} promoted the proteolytic activation of cotransfected procaspase-1, as well as the corresponding release of mature IL-1 into the medium, to a similar extent (FIG. 4, Panel A). In contrast, cotransfection with Rac1_{CA-F37L} was unable to promote the activation of caspase-1 and the release of IL-1. Therefore, we can conclude that Rac1-mediated signaling to JNK activation is not involved in caspase-1 activation. In contrast, caspase-1 activation seems to be limited to the Rac1-mediated control of the actin cytoskeleton. LIMK-1 participates in Rac1-mediated actin cytoskeletal reorganization by phosphorylating cofilin (Arber et al., 1998; Yang et al., 1998). Overexpression of constitutive active LIMK-1 could promote the activation of caspase-1, confirming the importance of the cytoskeleton in the activation process. Moreover, a dominant negative form of LIMK-1 could abrogate the Rac1 signaling pathway towards caspase-1 activation (FIG. 4, Panel B). The latter suggests that the signaling pathway from Rac1 towards caspase-1 activation could involve LIMK-1.

### Example 5

### Rac1 Controls the Asc-mediated Activation and Oligomerization of Caspase-1

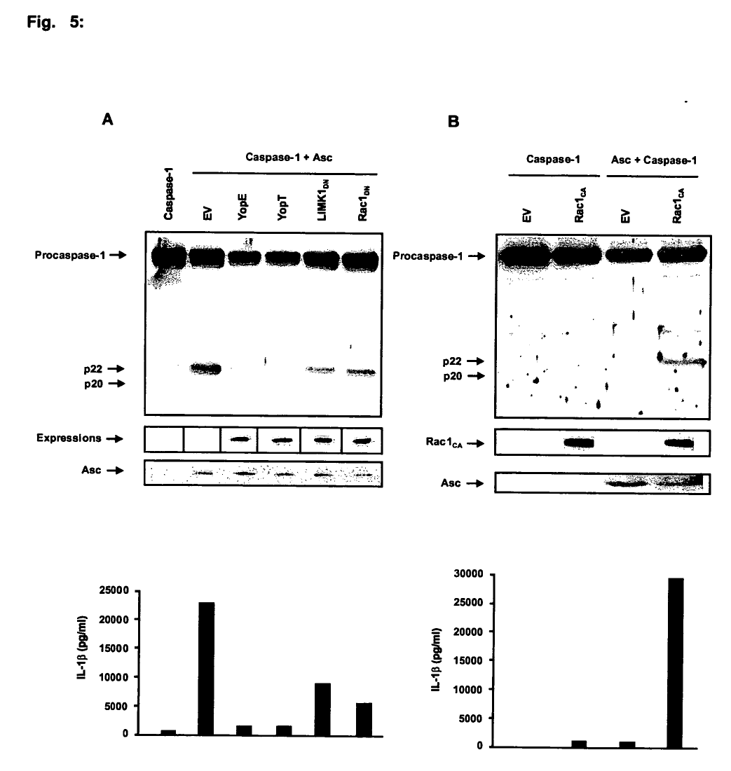

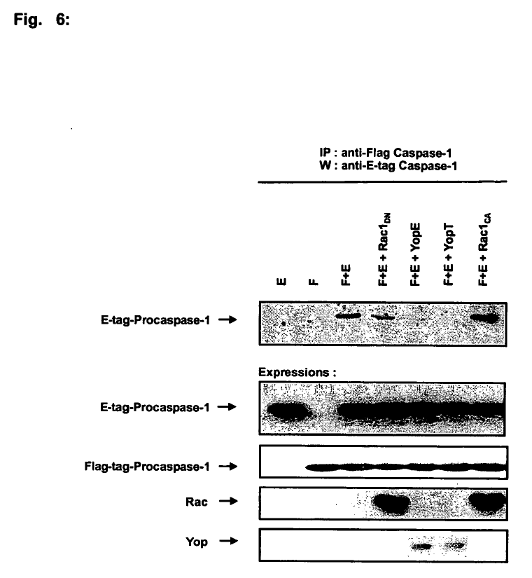

The molecular mechanism of caspase-1 activation is still largely unknown. The caspase recruitment domain- (CARD-) containing protein Asc has been shown to function as a caspase-1 activating adaptor protein by mediating the assembly of a caspase-1 signaling complex that promotes the activation of caspase-1 and the proteolytic maturation of proIL-1β (Srinivasula et al., 2002; Wang et al., 2002). To analyze if Rac1 and LIMK-1 can modulate the Asc-mediated activation of caspase-1, we cotransfected HEK293T cells with expression vectors for procaspase-1, Asc, proIL-1β and either YopE, YopT, LIMK-1_{DN} or Rac1_{DN}. Western blot analysis of caspase-1, as well as analysis of the production of bio-active IL-1β shows that Asc-mediated caspase-1 activation can be affected by Rac1_{DN} and LIMK-1_{DN}, as well as by the Yop effector proteins YopE and YopT (FIG. 5, Panel A). To analyze if Rac1_{CA} could promote the Asc-mediated caspase-1 activation, we co-expressed Asc or Rac1_{CA} with procaspase-1 to a level that does not lead to a significant activation of caspase-1. However, co-expression of constitutive-active Rac1_{CA} and Asc with procaspase-1 resulted in a strong activation of caspase-1 (FIG. 5, Panel B). These data suggest that Rac1 and LIMK-1 can regulate the Asc-mediated activation of caspase-1. We were unable to study the effect of Rac on Asc-induced caspase-1 oligomerization because overexpression of Asc led to the redistribution of caspase-1 oligomers to the insoluble cell fraction, which is consistent with the previously described formation of filament-like aggregates upon Asc overexpression (Masumoto et al., 2001). However, caspase-1 oligomerization can be forced by overexpression in HEK293T cells. To analyze further whether Rac1 modulates caspase-1 activation by affecting its oligomerization, we analyzed in a co-immunoprecipitation experiment whether Rac1, YopE and YopT could affect caspase-1 oligomerization by making use of two enzymatically inactive procaspase-1 molecules that were tagged with a different epitope-tag. As illustrated in FIG. 6, a dominant-negative Rac mutant (Rac1_{DN}), as well as YopE and YopT, could prevent the oligomerization of procaspase-1, with Yops being much more efficient then Rac1_{DN}. In contrast, coexpression of constitutive active Rac1 (Rac1_{CA}) could promote the formation of procaspase-1 oligomers. These experiments demonstrate that Rac1 modulates caspase-1 activation by affecting its oligomerization.

TABLE 1
Expression plasmids for Y. enterocolitica and Y. enterocolitica E40
and derivative strains

| | Strain or | |
|---|---|---|
| | plasmid | |
| Strain or | designation | |
| plasmid | in the | |
| code | paper | Relevant characteristics |
| Expression | | |
| plasmids | | |
| for | | |
| Yersinia | | |
| pLJM3 | YopE_{WT} | Wild-type yopE, expressed under the |
| | | control of its own promoter, and a |
| | | truncated sycE taken from pAPB26 |
| | | (Sauvonnet et al., 2002) |
| | | and cloned in the EcoRI and HindIII sites |
| | | of the low-copy vector pBBR1MCS-2 |
| | | (Denecker et al., 2001). Gift from Dr. |
| | | L. J. Mota. |
| pLJM4 | YopE_{M} | Catalytic mutant yopE, expressed under |
| | | the control of its own promoter, and a |
| | | truncated sycE taken from pNG7 (Orth |
| | | et al., 1999) and cloned in the EcoRI and |
| | | HindIII sites of the low-copy vector |
| | | pBBR1MCS-2 (Denecker et al., 2001). |
| | | Gift from Dr. L. J. Mota. |
| pYV40 | | |
| plasmids | | |
| E40(pYV40) | WT | Wild-type E40 (Fitzgerald, 2000). |
| E40(pMSK41) | YopP⁻ | yopP knockout (yopP₂₃ allele) of |
| | | E40(pYV40) (Goto et al., 1999). |
| E40(pSI4008) | YopH⁻ | yopH_{Δ1-352} knockout (yopH_{Δ1-352} |
| | | allele) of E40(pYV40) (Goto et al., |
| | | 1999). |
| E40(pAB406) | YopO⁻ | YopO_{Δ65-558} knockout (YopO_{Δ65-558} |
| | | allele) of E40(pYV40) (Goto et al., |
| | | 1999). |
| E40(pAB4052) | YopE⁻ | yopE₂₁ knockout (yopE₂₁ allele) of |
| | | E40(pYV40) (Goto et al., 1999). |
| E40(pAB408) | YopM⁻ | yopM₂₃ knockout (yopM₂₃ allele) of |
| | | E40(pYV40) (Goto et al., 1999). |
| E40(pIM409) | YopT⁻ | yopT₁₃₅ knockout (yopT₁₃₅ allele) of |
| | | E40(pYV40) (Howard et al., 1991). |
| E40(pIML421) | ΔHOPEMT | yopH_{Δ1-352}, YopO_{Δ65-558}, yopP₂₃, |
| | | yopE₂₁, yopM₂₃, yopT₁₃₅ knockout |
| | | (yopH_{Δ1-352}, YopO_{Δ65-558}, yopP₂₃, |
| | | yopE₂₁, yopM₂₃, yopT₁₃₅ allele) |
| | | of E40(pYV40) (Howard et al.,1991). |
| E40(pGD4005) | YopHP⁻ | yopH_{Δ1-352}, yopP₂₃ knockout |
| | | (yopH_{Δ1-352}, yopP₂₃ allele) |
| | | of E40(pYV40) |
| | | E40(pGD4005) was obtained by |
| | | conjugation of pSI4008 and pMSK7 |
| | | (Goto et al., 1999), the yopP mutator. |
| E40(pGD4003) | YopOP⁻ | YopO_{Δ65-558}, yopP₂₃ knockout |
| | | (yopO_{Δ65-558}, yopP₂₃ |
| | | allele) of E40(pYV40) |
| | | E40(pGD4003) was obtained by |
| | | conjugation of pMSK41 and pAB34 |
| | | (Goto et al., 1999), the yopO mutator. |
| E40(pAB407) | YopEP⁻ | yopE₂₁, yopP₂₃ knockout |
| | | (yopE₂₁, yopP₂₃ allele) of |
| | | E40(pYV40) |
| | | E40(pAB407) was obtained by |
| | | conjugation of pAB4052 and pMSK7 |
| | | (Goto et al., 1999), the yopP |
| | | mutator. Gift from Dr. A. Boland. |
| E40(pGD4004) | YopMP⁻ | yopM₂₃, yopP₂₃ knockout |
| | | (yopM₂₃, yopP₂₃ allele) of |
| | | E40(pYV40) |
| | | E40(pGD4004) was obtained by |
| | | conjugation of pAB408 and pMSK7 |
| | | (Goto et al., 1999), the yopP |
| | | mutator.. |
| E40(pGD4002) | YopTP⁻ | yopT₁₃₅, yopP₂₃ knockout |
| | | (yopT₁₃₅, yopP₂₃ allele) of |
| | | E40(pYV40) |
| | | E40(pGD4002) was obtained by |
| | | conjugation of pIM409 and pMSK7 |
| | | (Goto et al., 1999), the yopP |
| | | mutator. |
| E40(pAB407) | YopEP⁻ + | E40(pAB407), |
| (pLJM3) | YopE_{WT} | complemented with pLJM3. |
| E40(pAB407) | YopEP⁻ + | E40(pAB407), |
| (pLJM4) | YopE_{M} | complemented with pLJM4. |

### REFERENCES

Aepfelbacher, M., C. Trasak, G. Wilharm, A. Wiedemann, K. Trulzsch, K. Krauss, P. Gierschik, and J. Heesemann. 2003. Characterization of YopT effects on Rho GTPases in Yersinia enterocolitica-infected cells. J. Biol. Chem. 278:33217-23.
Andor, A., K. Trulzsch, M. Essler, A. Roggenkamp, A. Wiedemann, J. Heesemann, and M. Aepfelbacher. 2001. YopE of Yersinia, a GAP for Rho GTPases, selectively modulates Rac-dependent actin structures in endothelial cells. Cell Microbiol. 3:301-10.
Arber, S., F. A. Barbayannis, H. Hanser, C. Schneider, C. A. Stanyon, O. Bernard, and P. Caroni. 1998. Regulation of actin dynamics through phosphorylation of cofilin by LIM-kinase. Nature 393:805-9.
Bishop, A. L., and A. Hall. 2000. Rho GTPases and their effector proteins. Biochem. J. 348 Pt 2:241-55.
Black, D. S., and J. B. Bliska. 1997. Identification of p130Cas as a substrate of Yersinia YopH (Yop51), a bacterial protein tyrosine phosphatase that translocates into mammalian cells and targets focal adhesions. Embo. J. 16:2730-44.
Black, D. S., A. Marie-Cardine, B. Schraven, and J. B. Bliska. 2000. The Yersinia tyrosine phosphatase YopH targets a novel adhesion-regulated signaling complex in macrophages. Cell Microbiol. 2:401-14.
Cornelis, G. R. 2002. The Yersinia Ysc-Yop “Type III” weaponry. Nat. Rev. Mol. Cell Biol. 3:742-54.
Cornelis, G. R., A. Boland, A. P. Boyd, C. Geuijen, M. Iriarte, C. Neyt, M. P. Sory, and I. Stainier. 1998. The virulence plasmid of Yersinia, an antihost genome. Microbiol. Mol. Biol. Rev. 62:1315-1352.
Denecker, G., W. Declercq, C. A. Geuijen, A. Boland, R. Benabdillah, M. van Gurp, M. P. Sory, P. Vandenabeele, and G. R. Cornelis. 2001. Yersinia enterocolitica YopP-induced apoptosis of macrophages involves the apoptotic signaling cascade upstream of bid. J. Biol. Chem. 276:19706-14.
Desmedt, M., P. Rottiers, H. Dooms, W. Fiers, and J. Grooten. 1998. Macrophages induce cellular immunity by activating Th1 cell responses and suppressing Th2 cell responses. J. Immunol. 160:5300-8.
Fitzgerald, K. A., and L. A. O'Neill. 2000. The role of the interleukin-1/Toll-like receptor superfamily in inflammation and host defense. Microbes Infect. 2:933-43.
Goto, M., K. I. Katayama, F. Shirakawa, and I. Tanaka. 1999. Involvement of NF-κB p50/p65 heterodimer in activation of the human pro-interleukin-1β gene at two subregions of the upstream enhancer element. Cytokine 11:16-28.
Grosdent, N., I. Maridonneau-Parini, M. P. Sory, and G. R. Cornelis. 2002. Role of Yops and adhesins in resistance of Yersinia enterocolitica to phagocytosis. Infect. Immun. 70:4165-76.
Heyninck, K., D. De Valck, W. Vanden Berghe, W. Van Criekinge, R. Contreras, W. Fiers, G. Haegeman, and R. Beyaert. 1999. The zinc finger protein A20 inhibits TNF-induced NF-κB-dependent gene expression by interfering with an RIP- or TRAF2-mediated transactivation signal and directly binds to a novel NF-κB- inhibiting protein ABIN. J. Cell Biol. 145:1471-82.
Howard, A. D., M. J. Kostura, N. Thomberry, G. J. Ding, G. Limjuco, J. Weidner, J. P. Salley, K. A. Hogquist, D. D. Chaplin, R. A. Mumford, and et al. 1991. IL-1-converting enzyme requires aspartic acid residues for processing of the IL-1 beta precursor at two distinct sites and does not cleave 31- kDa IL-1 alpha. J. Immunol. 147:2964-9.
Iriarte, M., and G. R. Cornelis. 1998. YopT, a new Yersinia Yop effector protein, affects the cytoskeleton of host cells. Mol. Microbiol. 29:915-29.
Lamarche, N., N. Tapon, L. Stowers, P. D. Burbelo, P. Aspenstrom, T. Bridges, J. Chant, and A. Hall. 1996. Rac and Cdc42 induce actin polymerization and G1 cell cycle progression independently of p65PAK and the JNK/SAPK MAP kinase cascade. Cell 87:519-29.
Masumoto, J., S. Taniguchi, and J. Sagara. 2001. Pyrin N-terminal homology domain- and caspase recruitment domain-dependent oligomerization of ASC. Biochem. Biophys. Res. Commun. 280:652-5.
Mills, S. D., A. Boland, M. P. Sory, P. van der Smissen, C. Kerbourch, B. B. Finlay, and G. R. Cornelis. 1997. Yersinia enterocolitica induces apoptosis in macrophages by a process requiring functional type III secretion and translocation mechanisms and involving YopP, presumably acting as an effector protein. Proc. Natl. Acad. Sci. U.S.A. 94:12638-43.
Niwa, H., K. Yamamura, and J. Miyazaki. 1991. Efficient selection for high-expression transfectants with a novel eukaryotic vector. Gene 108:193-9.
Orth, K., L. E. Palmer, Z. Q. Bao, S. Stewart, A. E. Rudolph, J. B. Bliska, and J. E. Dixon. 1999. Inhibition of the mitogen-activated protein kinase kinase superfamily by a Yersinia effector. Science 285:1920-3.
Persson, C., N. Carballeira, H. Wolf-Watz, and M. Falhnan. 1997. The PTPase YopH inhibits uptake of Yersinia, tyrosine phosphorylation of p130Cas and FAK, and the associated accumulation of these proteins in peripheral focal adhesions. Embo. J. 16:2307-18.
Prepens, U., I. Just, C. von Eichel-Streiber, and K. Aktories. 1996. Inhibition of Fc epsilon-RI-mediated activation of rat basophilic leukemia cells by Clostridium difficile toxin B (monoglucosyltransferase). J. Biol. Chem. 271:7324-9.
Ruckdeschel, K., O. Mannel, K. Richter, C. A. Jacobi, K. Trulzsch, B. Rouot, and J. Heesemann. 2001. Yersinia outer protein P of Yersinia enterocolitica simultaneously blocks the nuclear factor-kappa B pathway and exploits lipopolysaccharide signaling to trigger apoptosis in macrophages. J. Immunol. 166:1823-31.
Sander, E. E., J. P. ten Klooster, S. van Delft, R. A. van der Kammen, and J. G. Collard. 1999. Rac down-regulates Rho activity: reciprocal balance between both GTPases determines cellular morphology and migratory behavior. J. Cell Biol. 147:1009-22.
Sauvonnet, N., I. Lambermont, P. van der Bruggen, and G. R. Cornelis. 2002a. YopH prevents monocyte chemoattractant protein 1 expression in macrophages and T-cell proliferation through inactivation of the phosphatidylinositol 3-kinase pathway. Mol. Microbiol. 45:805-15.
Shao, F., P. M. Merritt, Z. Bao, R. W. Innes, and J. E. Dixon. 2002. A Yersinia effector and a Pseudomonas avirulence protein define a family of cysteine proteases functioning in bacterial pathogenesis. Cell 109:575-88.
Sory, M. P., A. Boland, I. Lambermont, and G. R. Cornelis. 1995. Identification of the YopE and YopH domains required for secretion and internalization into the cytosol of macrophages, using the cyaA gene fusion approach. Proc. Natl. Acad. Sci. U.S.A. 92:11998-2002.
Srinivasula, S. M., J. L. Poyet, M. Razmara, P. Datta, Z. Zhang, and E. S. Alnemri. 2002. The PYRIN-CARD protein ASC is an activating adaptor for caspase-1. J. Biol. Chem. 277:21119-22.
Vandenabeele, P., W. Declercq, C. Libert, and W. Fiers. 1990. Development of a simple, sensitive and specific bioassay for interleukin-1 based on the proliferation of RPMI 1788 cells. Comparison with other bioassays for IL-1. J. Immunol. Methods 135:25-32.
Vasudevan, A., Y. Qian, A. Vogt, M. A. Blaskovich, J. Ohkanda, S. M. Sebti, and A. D. Hamilton. 1999. Potent, highly selective, and non-thiol inhibitors of protein geranylgeranyltransferase-I. J. Med. Chem. 42:1333-40.
Viboud, G. I., and J. B. Bliska. 2001. A bacterial type III secretion system inhibits actin polymerization to prevent pore formation in host cell membranes. Embo. J. 20:5373-82.
Von Pawel-Rammingen, U., M. V. Telepnev, G. Schmidt, K. Aktories, H. Wolf-Watz, and R. Rosqvist. 2000. GAP activity of the Yersinia YopE cytotoxin specifically targets the Rho pathway: a mechanism for disruption of actin microfilament structure. Mol. Microbiol. 36:737-48.
Wang, L., G. A. Manji, J. M. Grenier, A. Al-Garawi, S. Merriam, J. M. Lora, B. J. Geddes, M. Briskin, P. S. DiStefano, and J. Bertin. 2002. PYPAF7, a novel PYRIN-containing Apaf1-like protein that regulates activation of NF-kappa B and caspase-1-dependent cytokine processing. J. Biol. Chem. 277:29874-80.
Westwick, J. K., Q. T. Lambert, G. J. Clark, M. Symons, L. Van Aelst, R. G. Pestell, and C. J. Der. 1997. Rac regulation of transformation, gene expression, and actin organization by multiple, PAK-independent pathways. Mol. Cell Biol. 17:1324-35.
Wilkins, T. D., and D. M. Lyerly. 1996. Clostridium difficile toxins attack Rho. Trends Microbiol. 4:49-51.
Yang, N., O. Higuchi, K. Ohashi, K. Nagata, A. Wada, K. Kangawa, E. Nishida, and K. Mizuno. 1998. Cofilin phosphorylation by LIM-kinase 1 and its role in Rac-mediated actin reorganization. Nature 393:809-12.

1. A method of modulating caspase activation and/or activity comprising the step of administering one of a Yersinia outermembrane protein (Yop) effector protein, a Rho GTPase, and a LIMK-1.

2. A method of inhibiting oligomerization of caspase comprising the step of administering one of a Yersinia outermembrane protein (Yop) effector protein, a Rho GTPase, and a LIMK-1.

3. A method of treating a caspase related disease comprising the step of administering one of a Yersinia outermembrane protein (Yop) effector protein, a Rho GTPase, and a LIMK-1.

4. The method according to claim 1, wherein the caspase is caspase-1.

5. A method of inhibiting caspase-1 medicated cell death comprising the step of administering one of a Yersinia outermembrane protein (Yop) effector protein, a Rho GTPase, and a LIMK-1.

6. A method of inhibiting interleukin-1β (IL-1β) maturation and/or its release from cells comprising the step of administering one of a Yersinia outermembrane protein (Yop) effector protein, a Rho GTPase, and a LIMK-1.

7. A method of inhibiting interleukin-18 (IL-18) maturation and/or its release from cells comprising the step of administering one of a Yersinia outermembrane protein (Yop) effector protein, a Rho GTPase, and a LIMK-1.

8. The method according to claim 1, wherein the Yop is selected from the group consisting of YopE and YopT.

9. The method of claim 1, wherein the Rho GTPase is selected from the group consisting of RhoA, Rac1, or Cdc42.
